SEQUENCE LISTING (28 sequences)

SEQ ID NO: 1 - protein, 14 aa, Artificial Sequence
  Description of Artificial Sequence Synthetic peptide
RGDACXGDSGGPXV

SEQ ID NO: 2 - protein, 23 aa, Artificial Sequence
  Description of Artificial Sequence Synthetic peptide
AGYKPDEGKRGDACXGDSGGPXV

SEQ ID NO: 3 - protein, 23 aa, Artificial Sequence
  Description of Artificial Sequence Synthetic peptide
AGYKPDEGKRGDACEGDSGGPFV

SEQ ID NO: 4 - protein, 14 aa, Artificial Sequence
  Description of Artificial Sequence Synthetic peptide
RGDAXXGDSGGPXV

SEQ ID NO: 5 - protein, 23 aa, Artificial Sequence
  Description of Artificial Sequence Synthetic peptide
AGYKPDEGKRGDAXXGDSGGPXV

SEQ ID NO: 6 - protein, 23 aa, Artificial Sequence
  Description of Artificial Sequence Synthetic peptide
AGYKPDEGKRGDACEGDSGGPFV

SEQ ID NO: 7 - protein, 10 aa, Artificial Sequence
  Description of Artificial Sequence Synthetic peptide
EGKRGDACEG

SEQ ID NO: 8 - protein, 33 aa, Artificial Sequence
  Description of Artificial Sequence Synthetic polypeptide
AGYKPDEGKRGDACXGDSGGPXVMKSPFNNRWY

SEQ ID NO: 9 - protein, 33 aa, Artificial Sequence
  Description of Artificial Sequence Synthetic polypeptide
AGYKPDEGKRGDACEGDSGGPFVMKSPFNNRWY

SEQ ID NO: 10 - protein, 12 aa, Artificial Sequence
  Description of Artificial Sequence Synthetic peptide
DACXGDSGGPXV

SEQ ID NO: 11 - protein, 14 aa, Artificial Sequence
  Description of Artificial Sequence Synthetic peptide
RGDAXEGDSGGPFV

SEQ ID NO: 12 - protein, 23 aa, Artificial Sequence
  Description of Artificial Sequence Synthetic peptide
AGYKPDEGKRGDACXGDSGGPXV

SEQ ID NO: 13 - protein, 12 aa, Artificial Sequence
  Description of Artificial Sequence Synthetic peptide
DXCXGDSGGPXV

SEQ ID NO: 14 - protein, 10 aa, Artificial Sequence
  Description of Artificial Sequence Synthetic peptide
CEGDSGGPFV

SEQ ID NO: 15 - protein, 10 aa, Artificial Sequence
  Description of Artificial Sequence Synthetic peptide
CXGDSGGPXV

SEQ ID NO: 16 - protein, 4 aa, Artificial Sequence
  Description of Artificial Sequence Synthetic peptide
RGDA

SEQ ID NO: 17 - protein, 14 aa, Artificial Sequence
  Description of Artificial Sequence Synthetic peptide
RGDACEGDSGGPFV

SEQ ID NO: 18 - protein, 33 aa, Artificial Sequence
  Description of Artificial Sequence Synthetic polypeptide
DNMFCAGYKPDEGKRGDACEGDSGGPFVMKSPF

SEQ ID NO: 19 - protein, 33 aa, Artificial Sequence
  Description of Artificial Sequence Synthetic polypeptide
DNMFCAGYKPDEGKRGDACXGDSGGPXVMKSPF

SEQ ID NO: 20 - protein, 23 aa, Artificial Sequence
  Description of Artificial Sequence Synthetic peptide
AGYKPDEGKRGDAXEGDSGGPFV

SEQ ID NO: 21 - protein, 23 aa, Artificial Sequence
  Description of Artificial Sequence Synthetic peptide
AGYKPDEGKRGDAAEGDSGGPFV

SEQ ID NO: 22 - protein, 23 aa, Artificial Sequence
  Description of Artificial Sequence Synthetic peptide
AGYKPDEGKRGDAAEGDSGGPFV

SEQ ID NO: 23 - protein, 33 aa, Artificial Sequence
  Description of Artificial Sequence Synthetic polypeptide
DNMFXAGYKPDEGKRGDAXEGDSGGPFVMKSPF

SEQ ID NO: 24 - protein, 33 aa, Artificial Sequence
  Description of Artificial Sequence Synthetic polypeptide
DNMFXAGYKPDEGKRGDAXXGDSGGPXVMKSPF

SEQ ID NO: 25 - protein, 33 aa, Artificial Sequence
  Description of Artificial Sequence Synthetic polypeptide
DNMFAAGYKPDEGKRGDAAEGDSGGPFVMKSPF

SEQ ID NO: 26 - protein, 33 aa, Artificial Sequence; identical to SEQ ID NO: 25
  Description of Artificial Sequence Synthetic polypeptide

SEQ ID NO: 27 - protein, 33 aa, Artificial Sequence; identical to SEQ ID NO: 9
  Description of Artificial Sequence Synthetic polypeptide

SEQ ID NO: 28 - protein, 23 aa, Artificial Sequence
  Description of Artificial Sequence Synthetic peptide
AGYKPDEGKRGDASEGDSGGPFV

(19) United States
(12) Patent Application Publication
(10) Pub. No.: US 2011/0117075 A1
(43) Pub. Date: May 19, 2011

(54) THROMBIN DERIVED PEPTIDES FOR SMOOTH MUSCLE RELAXATION

(75) Inventors: Darrell H. Carney, Dickinson, TX (US); Barbara Olszewska-Pazdrak, League City, TX (US); Theresa W. Fossum, College Station, TX (US)

(73) Assignee: ORTHOLOGIC CORP.

(21) Appl. No.: 12/934,424

(22) PCT Filed: Mar. 24, 2009
(86) PCT No.: PCT/US09/01819

§ 371 Date: Jan. 7, 2011

(30) Foreign Application Priority Data
Mar. 26, 2008 (US) 61/070877

(51) Int. Cl. A61K 38/48; A61P 1/00; A61P 21/02

(52) U.S. Cl. 424/94.64

(57) ABSTRACT

Agonists of a non-proteolytically activated thrombin receptor, and more particularly, thrombin peptide derivatives, can be used in methods to cause smooth muscle relaxation. Compositions comprising thrombin peptide derivatives can be administered to a subject with a disease or disorder that can be ameliorated by relaxation of smooth muscle. Such compositions can also be administered to a subject to facilitate medical, diagnostic or surgical procedures.

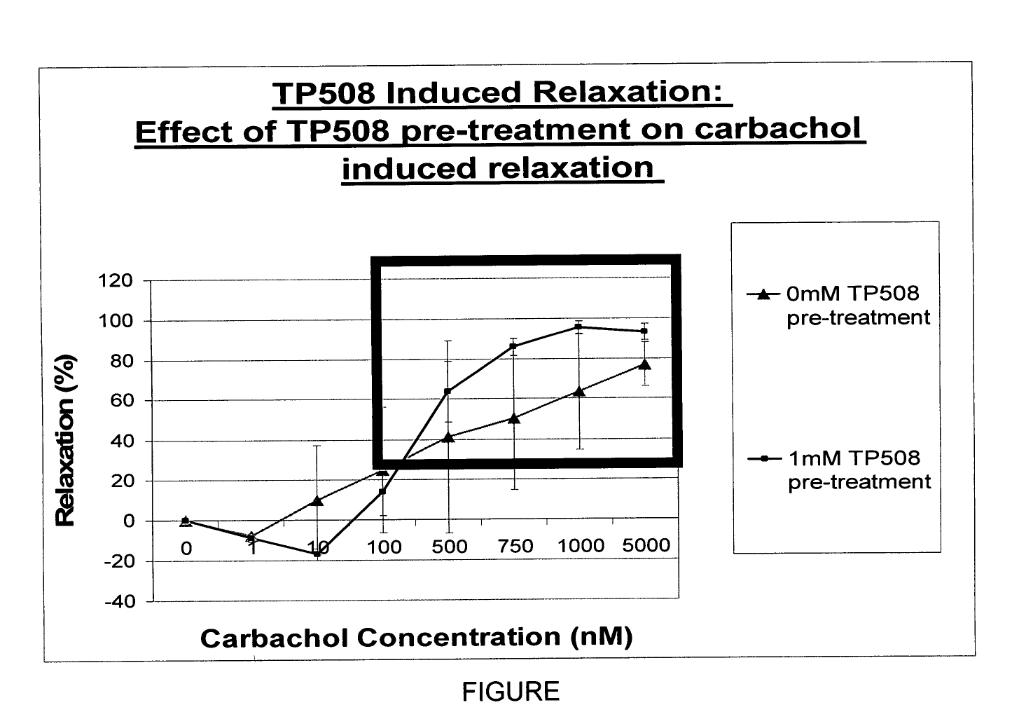

### RELATED APPLICATION

This application claims the benefit of U.S. Provisional Application No. 61/070,877, filed on Mar. 26, 2008. The entire teachings of the above application are incorporated herein by reference.

### BACKGROUND OF THE INVENTION

Smooth muscle is involuntary, nonstriated muscle. Smooth muscle is classified both functionally and anatomically. When classified functionally, it is broken into two groups: multi-unit, and single-unit smooth muscle. Briefly, multi-unit smooth muscle is activated by nerves which cause contraction of independent muscle units, and thus is not spontaneously active. Examples of multi-unit muscle may be found in large arteries and veins, the urinary bladder, and the iris and ciliary muscles of the eye. In contrast to multi-unit smooth muscle, single-unit smooth muscle is usually spontaneously active, and contains tight junctions which facilitate the contraction of the muscle as a single unit. Examples of single-unit smooth muscle may be found in the vasculature (small veins, small arteries, and arterioles), the bile duct, and the walls of the gastrointestinal and urinogenital systems (gut, ureter, and uterus).

When smooth muscle is anatomically classified, it is generally divided into two groups: vascular and non-vascular. Briefly, vascular smooth muscle consists only of blood vessels (arteries, arterioles, and veins), whereas non-vascular muscle includes all other types of smooth muscle [e.g., gastrointestinal (stomach, duodenum, ileum, jejunum, caecum, and colon), spleen, trachea/bronchus, seminal vesicle, ductus deferens, corpus cavernosum, biliary tract, ureter, and uterus].

Non-vascular smooth muscle is present in numerous organ systems throughout the body, and has a vital role in the physiological function of these systems. For example, airway smooth muscle plays a critical role in constriction and dilation of bronchi. In the gastrointestinal tract, the sphincter of Oddi, a smooth muscle connection between the bile duct and duodenum, provides tonic contraction which serves to prevent reflux of duodenal contents into the pancreatic and bile ducts, and promotes filling of the gall bladder. In addition, esophageal (sphincters and body), intestinal and colonic motility is regulated by smooth muscle. Smooth muscle of the bladder body, bladder base, and proximal urethra plays an important role in urological function.

A variety of significant clinical disorders occur, which involve contraction, spasm, or failure to achieve the necessary relaxation of smooth muscle. Examples of such disorders include airway obstruction (i.e., chronic bronchitis, acute bronchitis and emphysema), bladder dysfunction, and gastrointestinal muscle spasm (i.e., irritable bowel syndrome, achalasia, dumping disorders). Thus, a clinical need exists for pharmacological agents which can treat, prevent, or reduce the probability of developing such disorders by inducing relaxation of the affected smooth muscle.

### SUMMARY OF THE INVENTION

NPAR agonists, and in particular, thrombin peptide derivatives, can be used for the treatment, prevention, or reduction in probability of developing, disorders associated with contraction or spasm of smooth muscle, such as airway obstruction, gastrointestinal spasm, bladder dysfunction, urge urinary incontinence, angina, coronary vasospasm, subarachnoid hemorrhage, pre-term labor, intrauterine growth restriction, Raynaud's disease, non-occlusive mesenteric ischemia, anal fissure, achalasia, migraine, ischemic muscle injury associated with smooth muscle spasm, vasculopathy, emphysema, acute bronchitis, chronic bronchitis, acute respiratory failure, irritable bowel syndrome, rapid gastric emptying and diarrhea.

The invention is also directed to the use of NPAR agonists, and in particular, thrombin peptide derivatives, to alleviate smooth muscle contraction and spasm, and thus facilitate procedures involving diagnostic instrumentation such as endoscopy and bronchoscopy.

This invention includes methods for relaxing airway smooth muscle by administering a therapeutically effective amount of an NPAR agonist, and in particular, a thrombin peptide derivative, to a subject. The invention is also directed to a method for treatment, prevention, or reduction in the probability of developing respiratory disorders by administering a therapeutically effective amount of a thrombin peptide derivative to a subject. Respiratory disorders include, for instance, emphysema, acute bronchitis, chronic bronchitis, and acute respiratory failure.

The invention also includes methods for relaxing gastrointestinal smooth muscle, bladder smooth muscle, or uterine smooth muscle by administering a therapeutically effective amount of an NPAR agonist, and in particular, a thrombin peptide derivative, to a subject in need of relaxation of the smooth muscle.

The invention is also directed to methods wherein a composition comprising one or more NPAR agonists is administered to a subject by oral, sublingual, intravenous, topical, intramuscular, intranasal, or another route of delivery, to effect smooth muscle relaxation in the female reproductive tract, in the male reproductive tract, in the gastrointestinal system, or in the blood vessels.

### BRIEF DESCRIPTION OF THE DRAWINGS

The FIGURE is a graph of data from measurements on rings of rat aorta to assess the relaxant effect of TP508.

### DETAILED DESCRIPTION OF THE INVENTION

Compounds which stimulate a non-proteolytically activated thrombin receptor (NPAR) are said to be NPAR agonists. One such NPAR is a high-affinity thrombin receptor present on the surface of most cells. This NPAR component is largely responsible for high-affinity binding of thrombin, proteolytically inactivated thrombin, and thrombin derived peptides to cells. This NPAR appears to mediate a number of cellular signals that are initiated by thrombin independent of its proteolytic activity (see Sower, et. al., Experimental Cell Research 247:422 (1999)). This NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

Smooth muscle is, found, for example, in the gastrointestinal system, the respiratory tract, the bladder and ureters, the bile duct, the uterus, the female and male reproductive tracts, the blood vessels, the iris and ciliary muscle of the eye, the corpus cavernosa, and the piloerector muscles.

One or more NPAR agonists, and in particular, one or more thrombin peptide derivatives, can be used in methods to relax both vascular and non-vascular smooth muscle. Compositions comprising NPAR agonists can be administered to a subject in need of smooth muscle relaxation to treat, prevent, or reduce the probability of developing those conditions which result from, or involve, contraction of smooth muscle, for example, constriction of a sphincter. NPAR agonists can be administered to subjects who can benefit from therapeutic intervention causing complete or partial smooth muscle relaxation. NPAR agonists can be administered to subjects, (e.g., human patients) at risk for developing a disorder associated with smooth muscle contraction, to reduce the probability of developing the disorder. For example, treatment can cause a reduction in the probability of developing the disorder by up to 20, 30, 40, 50, 60, 70, 80, or 90 percent. Treatment can in some cases, delay the development of a disorder, reduce symptoms, or delay severity of symptoms.

Part of the invention relates to the administration of a therapeutically effective amount of an NPAR agonist to a subject to relax smooth muscle of the respiratory system, that is, the smooth muscle lining the bronchi or tracheal region. NPAR agonists can be administered as therapeutic agents for the treatment of, prevention of, or reduction in the probability of developing, respiratory disorders. The term “respiratory disorder” refers to any impairment of lung function which involves constriction of airways and changes in blood gas levels or lung function.

For example, airway obstruction constitutes a respiratory disorder which can occur as a result of acute pulmonary impairment or obstructive lung disease. Severe airway obstruction may ultimately result in life-threatening respiratory failure. Airway obstruction can occur with emphysema and acute or chronic bronchitis.

Acute respiratory failure may be a consequence of airway constriction secondary to pneumonia, thromboembolism, left ventricular failure or pneumothorax. Acute respiratory failure may also result from ventilation-perfusion imbalance. In one embodiment, one or more NPAR agonists can be delivered directly into the lungs by bronchoscopic instrument. This mode of administration permits titration of dose, and eliminates side effects of systemic therapy. This mode of administration also facilitates endoscopy by suppressing the cough reflex and associated bronchospasm. A complication of bronchoscopy, and thus an impediment to the successful completion of the procedure, is bronchospasm. NPAR agonists can also be used to relax airway smooth muscle and to eliminate bronchoscopy-induced bronchospasm.

Other embodiments of the invention relate to the administration of a therapeutically effective amount of an NPAR agonists to a subject to relax gastrointestinal smooth muscle. The term “gastrointestinal smooth muscle” refers to smooth muscle which is contained in any region of the gastrointestinal tract, including the sphincters that control the flow of contents of the gastrointestinal tract. Such regions include, but are not limited to, the esophagus, duodenum, sphincter of Oddi, biliary tract, ileum, sigmoid colon, pancreatic duct and common bile duct. Thrombin derivative peptides can be used for the treatment or prevention of gastrointestinal disorders. Disorders of the gastrointestinal tract include, for example, achalasia (spasm of the lower esophageal sphincter), diarrhea, rapid gastric emptying (dumping syndrome), and irritable bowel syndrome.

Additional embodiments of the invention relate to the administration of NPAR agonists to reduce or alleviate contraction or spasm of gastrointestinal smooth muscle, and thus facilitate successful completion of endoscopic procedures. Contraction or spasm of gastrointestinal smooth muscle imposes a technical obstacle which must frequently be overcome in order to enable the clinician to successfully perform endoscopic procedures.

The term “endoscopic procedures” refers to those diagnostic procedures which utilize an instrument which is introduced into the gastrointestinal tract to provide direct visualization of the gastrointestinal tract, for examination and/or therapeutic purposes. Such purposes include direct visualization, biopsy, access to the common bile duct, fluid aspiration and removal of foreign bodies, polyps, and other lesions. An example of a particular endoscopic procedure is esophagogastro-duodenoscopy, which is utilized for examination of the esophageal lumen, stomach and duodenum. Another example, endoscopic retrograde cholangiopancreatography (ERCP), allows visualization of the pancreatic duct, common bile duct and the entire biliary tract, including the gall bladder. Further examples of endoscopic procedures are colonoscopy and sigmoidoscopy.

Other medical procedures for diagnostic or therapeutic purposes or for surgery may require the insertion of other medical devices or instruments. For endoscopy or for other medical procedures or therapy, the relaxation of one or more sphincters in a subject may be desirable. The sphincters to be relaxed, partially or completely, by administration of an NPAR agonist include, for example, the cardioesophageal sphincter, the gastroesophageal sphincter, the palatopharyngeal sphincter, the pharyngoesophageal sphincter, the pyloric sphincter, the internal rectal sphincter, the sphincter urethrae, the sphincter oculi, the sphincter pupillae, the sphincter oris, the sphincter iridis, the sphincter of Oddi, Giordano's sphincter, O'Beirne's sphincter, Nélaton's sphincter, Lütkens' sphincter, the sphincter of Boyden, the sphincter vaginae, the sphincter vesicae, and the tubal sphincter.

Another embodiment of the claimed invention relates to the administration of a therapeutically effective amount of an NPAR agonist, or more particularly, a thrombin peptide derivative, to relax bladder smooth muscle. Bladder smooth muscle includes that of the bladder base, bladder body and proximal urethra. In addition, NPAR agonists can be used for the treatment of bladder dysfunction disorders which benefit from relaxation of bladder smooth muscle. Such disorders include, but are not limited to, problems with bladder filling, volume and continence.

In addition, NPAR agonists can be administered to cause relaxation of urethral and bladder base smooth muscle, and thus, facilitate cystoscopic examination of the urinary tract. The term “cystoscopic examination” refers to the introduction of a fiber optic instrument through the urethra and into the bladder, to achieve visualization of the interior of the urethra and bladder for diagnostic and therapeutic purposes.

Further embodiments of the invention include the administration of a therapeutically effective amount of one or more NPAR agonist to relax uterine smooth muscle. Increased contractility of uterine smooth muscle precipitates premature labor. Thus, an additional embodiment of the invention relates to the administration of thrombin derivative peptides for the treatment or prevention of premature labor.

NPAR agonists can also be used to relax fallopian tube smooth muscle. Fallopian tube smooth muscle plays a role in the transport of the egg to the uterus. Thus, one or more NPAR agonists can be used to regulate ovum transport, or to facilitate laparoscopic examination of the fallopian tubes, or to facilitate fertilization procedures.

Additional embodiments of the invention relate to the administration of a thrombin peptide derivative, for example, as part of a pharmaceutical composition, comprising a pharmaceutically acceptable carrier, to achieve any of the physiological effects discussed herein.

Any of the NPAR agonists, and more specifically, compositions comprising thrombin peptide derivatives can be administered to a subject by an appropriate route, for example, by intranasal, oral, enteral, topical, sublingual, rectal, intramuscular, intravenous, or subcutaneous means. Medical instrumentation to be used for delivery of NPAR agonists (and which can be used also for other therapeutic and/or diagnostic purposes) include, but are not limited to, bronchoscopic, endoscopic, laporascopic, and cystoscopic instruments.

A thrombin receptor binding domain is defined as a polypeptide or portion of a polypeptide which directly binds to the thrombin receptor and/or competitively inhibits binding between high-affinity thrombin receptors and alpha-thrombin. In one embodiment, the thrombin receptor binding domain or portion thereof comprises the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the amino acid sequence Glu-Gly-Lys-Arg-Gly-Asp-Ala-Cys-Glu-Gly (SEQ ID NO:7).

NPAR agonists of the present invention include thrombin peptide derivatives, modified thrombin peptide derivatives and thrombin peptide derivative dimers as disclosed herein.

### Thrombin Peptide Derivatives

Among NPAR agonists are thrombin peptide derivatives, which are analogs of thrombin that have an amino acid sequence derived at least in part from that of thrombin and are active at a non-proteolytically activated thrombin receptor. Thrombin peptide derivatives include, for example, peptides that are produced by recombinant DNA methods, peptides produced by enzymatic digestion of thrombin, and peptides produced synthetically, which can comprise amino acid substitutions compared to thrombin, and/or modified amino acid residues, especially at the termini.

NPAR agonists of the present invention include thrombin derivative peptides described in U.S. Pat. Nos. 5,352,664 and 5,500,412. In one embodiment, the NPAR agonist of the present invention is a thrombin peptide derivative or a physiologically functional equivalent, i.e., a polypeptide with no more than about fifty amino acid residues, preferably no more than about thirty amino acid residues and having sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR.

The thrombin peptide derivatives or modified thrombin peptide derivatives described herein preferably have from about 12 to about 23 amino acid residues, more preferably from about 19 to about 23 amino acid residues.

In another embodiment, the NPAR agonist of the present invention is a thrombin peptide derivative comprising a moiety represented by Structural Formula (I):

Asp-Ala-R (I).

R is a serine esterase conserved domain. Serine esterases (e.g., trypsin, thrombin, chymotrypsin and the like) have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in particulars which do not affect the function of the thrombin receptor binding domain or the serine esterase conserved amino acid sequence. Such particulars may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing 4-12 of the N-terminal amino acid residues of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁ is either Ala or Ser; X₂ is either Glu or Gln; and X₃ is Phe, Met, Leu, His, or Val.

In one embodiment, the serine esterase conserved sequence comprises the amino acid sequence Cys-Glu-Gly-Asp-Ser-Gly-Gly-Pro-Phe-Val (SEQ ID NO:14) or a C-terminal truncated fragment of a polypeptide having the amino acid sequence of SEQ ID NO:14. It is understood, however, that zero, one, two or three amino acid residues in the serine esterase conserved sequence can differ from the corresponding amino acid in SEQ ID NO:14. Preferably, the amino acid residues in the serine esterase conserved sequence which differ from the corresponding amino acid in SEQ ID NO:14 are conservative substitutions, and are more preferably highly conservative substitutions. A “C-terminal truncated fragment” refers to a fragment remaining after removing an amino acid residue or block of amino acid residues from the C-terminus, said fragment having at least six and more preferably at least nine amino acid residues.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acid residues, preferably at least nine amino acid residues.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO:1). X₁ and X₂ are as defined above. The thrombin peptide derivative can comprise the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or an N-terminal truncated fragment thereof, provided that zero, one, two or three amino acid residues at positions 1-9 in the thrombin peptide derivative differ from the amino acid residue at the corresponding position of SEQ ID NO:6. Preferably, the amino acid residues in the thrombin peptide derivative which differ from the corresponding amino acid residues in SEQ ID NO:6 are conservative substitutions, and are more preferably highly conservative substitutions. An “N-terminal truncated fragment” refers to a fragment remaining after removing an amino acid residue or block of amino acid residues from the N-terminus, preferably a block of no more than six amino acid residues, more preferably a block of no more than three amino acid residues.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a substituted or unsubstituted aliphatic group comprising up to 10 carbon atoms, or Rₐ and R_{b}, taken together with the nitrogen to which they are bonded, form a C₃-C₁₀ non-aromatic heterocyclic group, and said N-terminal acyl group is represented by R_{c}C(O)—, wherein R_{c} is hydrogen, a substituted or unsubstituted aliphatic group comprising up to 10 carbon atoms, or a C₃-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivatives comprising the amino acid sequences SEQ ID NO: 6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acid residues at positions 1-9 and 14-23 in the thrombin peptide derivative can differ from the corresponding amino acid in SEQ ID NO:6. It is also understood that zero, one, two or three amino acid residues at positions 1-14 and 19-33 in the thrombin peptide derivative can differ from the corresponding amino acid in SEQ ID NO:18. Preferably, the amino acid residues in the thrombin peptide derivative which differ from the corresponding amino acid in SEQ ID NO:6 or SEQ ID NO:18 are conservative substitutions, and are more preferably highly conservative substitutions. Alternatively, an N-terminal truncated fragment of the thrombin peptide derivative having at least fourteen amino acid residues or a C-terminal truncated fragment of the thrombin peptide derivative having at least eighteen amino acid residues is a thrombin peptide derivative to be used as an NPAR agonist.

A “C-terminal truncated fragment” refers to a fragment remaining after removing an amino acid or block of amino acid residues from the C-terminus. An “N-terminal truncated fragment” refers to a fragment remaining after removing an amino acid residue or block of amino acid residues from the N-terminus. It is to be understood that both C-terminal truncated fragments and N-terminal truncated fragments can optionally be amidated at the C-terminus and/or acylated at the N-terminus.

A preferred thrombin peptide derivative for use in the disclosed methods comprises the polypeptide Ala-Gly-Tyr-Lys-Pro-Asp-Glu-Gly-Lys-Arg-Gly-Asp-Ala-Cys-Xᵢ-Gly-Asp-Ser-Gly-Gly-Pro-X₂-Val (SEQ ID NO:2). Another preferred thrombin peptide derivative for use in the disclosed method comprises the polypeptide Asp-Asn-Met-Phe-Cys-Ala-Gly-Tyr-Lys-Pro-Asp-Glu-Gly-Lys-Arg-Gly-Asp-Ala-Cys-Xᵢ-Gly-Asp-Ser-Gly-Gly-Pro-X₂-Val-Met-Lys-Ser-Pro-Phe (SEQ ID NO:19). X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acid residues, or C-terminal truncated fragments of these preferred thrombin peptide derivatives, the C-terminal truncated fragments having at least eighteen amino acid residues, can also be used in the disclosed methods.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acid residues, or C-terminal truncated fragments of TP508 (or deamide TP508), the C-terminal truncated fragments having at least eighteen amino acid residues.

As used herein, a “conservative substitution” in a polypeptide is the replacement of an amino acid with another amino acid that has the same net electronic charge and approximately the same size and shape. Amino acid residues with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acid residues with phenyl or substituted phenyl groups in their side chains are considered to have about the same size and shape. Listed below are five groups of amino acids. Replacing an amino acid residue in a polypeptide with another amino acid residue from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

### Modified Thrombin Peptide Derivatives

In one embodiment of the invention, the NPAR agonists are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, and an S-protected cysteine. Preferably, cysteine is replaced with alanine or serine. The modified thrombin peptide derivatives have about the same biological activity as the unmodified thrombin peptide derivatives.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acids. More specifically, the thrombin peptide derivative comprises the polypeptide Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val (SEQ ID NO:20), or a fragment thereof comprising amino acid residues10-18 of SEQ ID NO:20. Even more specifically, the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5. Xaa is alanine, glycine, serine or an S-protected cysteine. X₁ is Glu or Gln and X₂ is Phe, Met, Leu, His or Val. In one embodiment, X₁ is Glu, X₂ is Phe, and Xaa is Ala. In another embodiment, Xᵢ is Glu, X₂ is Phe, and Xaa is Ser. One example of a thrombin peptide derivative of this type is the polypeptide Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22), wherein H is a hydrogen atom of alanine indicating no modification at the N-terminus, and NH₂ indicates amidation at the C-terminus as —C(O)NH₂. Zero, one, two or three amino acids in the thrombin peptide derivative differ from the amino acid at the corresponding position of SEQ ID NO:4, 20, 5, 21 or 22, provided that Xaa is alanine, glycine, serine and an S-protected cysteine. Preferably, the difference is conservative.

In another specific embodiment, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe (SEQ ID NO:23), or a fragment thereof comprising amino acids 6-28. More preferably, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe (SEQ ID NO:24), or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X₁ is Glu, X₂ is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe (SEQ ID NO:25). A further example of a thrombin peptide derivative of this type is the polypeptide H-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26), wherein H is a hydrogen atom of aspartic acid indicating no modification at the N-terminus, and NH₂ indicates amidation at the C-terminus as —C(O)NH₂. Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g, cysteine-S—S-t-Bu). Preferably the protective group is an alkyl thioether. More preferably, the S-protected cysteine is an S-methyl cysteine. Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

The thrombin peptide derivatives or the modified thrombin peptide derivatives of the present invention can be mixed with a dimerization inhibitor for the preparation of a pharmaceutical composition comprising the thrombin peptide derivatives or the modified thrombin peptide derivatives of the present invention. Dimerization inhibitors can include chelating agents and/or thiol-containing compounds. An antioxidant can also be used in combination with the chelating agent and/or the thiol-containing compound.

A “chelating agent,” as used herein, is a compound having multiple sites (two, three, four or more) which can simultaneously bind to a metal ion or metal ions such as, for example, lead, cobalt, iron or copper ions. The binding sites typically comprise oxygen, nitrogen, sulfur or phosphorus. For example, salts of EDTA (ethylenediaminetetraacetic acid) can form at least four to six bonds with a metal ion or metal ions via the oxygen atoms of four acetic acid moieties (—CH₂C(O)O⁻) and the nitrogen atoms of ethylenediamine moieties (>N—CH₂—CH₂—N<) of EDTA. It is understood that a chelating agent also includes a polymer which has multiple binding sites to a metal or metal ions. Preferably, a chelating agent of the invention is non-toxic and does not cause unacceptable side effects at the dosages of pharmaceutical composition being administered in the methods of the invention. As a chelating agent of the invention, a copper-chelating agent is preferable. A “copper-chelating agent” refers to a chelating agent which can bind to a copper ion or copper ions. Examples of the copper-chelating agent include ethylenediaminetetraacetic acid (EDTA), penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine (TEPA), triethylenetetraamine and tris(2-carboxyethyl) phosphine (TCEP). Additional chelating agents are diethylenetriaminepentacetic acid (DTPA) and bathophenanthroline disulfonic acid (BPADA). EDTA is a preferred chelating agent. Typical amounts of a chelating agent present in the pharmaceutical compositions of the instant invention are in a range of between about 0.00001% and about 0.1% by weight, preferably between about 0.0001% and about 0.05% by weight.

A “pharmaceutically acceptable thiol-containing compound” as used herein is a compound which comprises at least one thiol (—SH) group and which does not cause unacceptable side effects at the dosages which are being administered. Examples of a pharmaceutically acceptable thiol-containing compound include thioglycerol, mercaptoethanol, thioglycol, thiodiglycol, cysteine, thioglucose, dithiothreitol (DTT) and dithio-bis-maleimidoethane (DTME). Typically, between about 0.001% and about 5% by weight, preferably between about 0.05% and about 1.0% by weight of a pharmaceutically acceptable thiol-containing compound is present in the pharmaceutical compositions of the invention.

An “antioxidant,” as used herein, is a compound which is used to prevent or reduce an oxidation reaction caused by an oxidizing agent such as oxygen. Examples of antioxidants include tocopherol, cystine, methionine, glutathione, tocotrienol, dimethyl glycine, betaine, butylated hydroxyanisole, butylated hydroxytoluene, vitamin E, ascorbic acid, ascorbyl palmitate, thioglycolic acid and antioxidant peptides such as, for example, turmerin. Typically, between about 0.001% and about 10% by weight, preferably between about 0.01% and about 5%, more preferably between about 0.05% and about 2.0% by weight of an antioxidant is present in the pharmaceutical compositions of the invention.

It is understood that certain chelating agents or thiol-containing compounds may also function as antioxidants, for example, tris(2-carboxyethyl) phosphine, cysteine or dithiothreitol. Other types of commonly used antioxidants, however, do not contain a thiol group. It is also understood that certain thiol-containing compounds may also function as a chelating agent, for example, dithiothreitol. Other types of commonly used chelating agents, however, do not contain a thiol group. It is also understood that the pharmaceutical compositions of the instant invention can comprise more than one chelating agent, thiol-containing compound or antioxidant. That is, for example, a chelating agent can be used either alone or in combination with one or more other suitable chelating agents.

### Thrombin Peptide Derivative Dimers

In some aspects of the present invention, the NPAR agonists of the methods are thrombin peptide derivative dimers. The dimers essentially do not revert to monomers and still have about the same biological activity as the thrombin peptide derivative monomers described above. A “thrombin peptide derivative dimer” is a molecule comprising two thrombin peptide derivatives (polypeptides) linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprise the thrombin peptide derivatives described above. Specifically, thrombin peptide derivatives have fewer than about fifty amino acids, preferably about thirty-three or fewer amino acids. The thrombin peptide derivative dimers described herein are formed from polypeptides typically having at least six amino acids and preferably from about 12 to about 33 amino acid residues, and more preferably from about 12 to about 23 amino acid residues. Thrombin peptide derivative monomer subunits of the dimers have sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6)) so that NPAR is activated.

In a specific embodiment, each of the two thrombin peptide derivatives (monomers) of a dimer comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. More specifically, a polypeptide monomer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or a fragment thereof comprising amino acid residues 10-18 of SEQ ID NO: 5. Even more specifically, a polypeptide monomer comprises the polypeptide Ala-Gly-Tyr-Lys-Pro-Asp-Glu-Gly-Lys-Arg-Gly-Asp-Ala-Cys-Xᵢ-Gly-Asp-Ser-Gly-Gly-Pro-X₂-Val (SEQ ID NO:2), or a fragment thereof comprising amino acid residues 10-18 of SEQ ID NO:2. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3), wherein H signifies a hydrogen atom of alanine indicating no modification at the N-terminus, and NH₂ signifies amidation at the C-terminus as —C(O)NH₂. Zero, one, two or three amino acid residues in the polypeptide differ from the amino acid residue at the corresponding position of SEQ ID NO:6, 1, 2, or 3. Preferably, the difference is conservative.

One example of a thrombin peptide derivative dimer of the present invention is represented by Formula (IV):

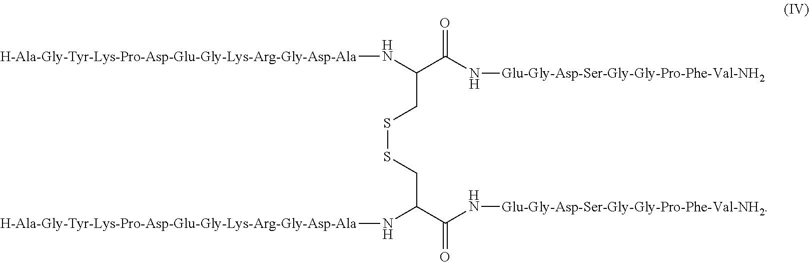

In another specific embodiment, each of the two thrombin peptide derivatives (monomers) of a dimer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27), or a C-terminal truncated fragment thereof having at least twenty-three amino acid residues. More preferably, a polypeptide comprises Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr (SEQ ID NO:8), or a C-terminal truncated fragment thereof comprising at least twenty-three amino acid residues. X₁ is Glu or Gln and X₂ is Phe, Met, Leu, His or Val. Preferably X₁ is Glu, and X₂ is Phe. One example of a polypeptide of this type is the polypeptide Ala-Gly-Phe-Val-Met-Lys-Ser-Pro-Phe-Asn-Asn-Arg-Trp-Tyr (SEQ ID NO:27). A further example of a polypeptide of this type is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:9), wherein H signifies a hydrogen atom of alanine indicating no modification at the N-terminus, and NH₂ indicates amidation at the C-terminus —C(O)NH₂. Zero, one, two or three amino acid residues in the polypeptide differ from the amino acid residue at the corresponding position of SEQ ID NO:27, 28 or 29. Preferably, the difference is conservative.

Methods of Treatment with NPAR Agonists

A “therapeutically effective amount” is the quantity of the NPAR agonist that results in relaxation of smooth muscle compared to untreated or sham-treated controls. NPAR agonists can effectively reduce contraction or spasm of smooth muscle.

Measurement of the effectiveness of smooth muscle relaxant compositions can be determined by measurement of the smooth muscle relaxation response, in a manner analogous to contractile response. See e.g., Wang, Poon and Pang, J. Pharm. Exp. Ther. 265:112-119, 1993. Briefly, tissue is first contracted with an agonist such as phenylephrine, usually at a concentration which results in 90% of the tissue's maximum contractile response (referred to EC₉₀). At the steady state phase of the contractile response to the agonist, a cumulative dose-response curve for the smooth muscle relaxant may be determined. For each concentration of relaxant, a steady state is achieved and the relaxation response measured. The effective concentration (EC) of a relaxant is the concentration that produces relaxation of a contraction previously induced by an agonist. For example, the EC₅₀ of a relaxant is the effective concentration of the relaxant required to relax a pre-contracted muscle to 50% of its maximum contractile response.

The amount of the NPAR agonist administered will depend on the degree of severity of smooth muscle contraction or the extent of smooth muscle relaxation desired, and will further depend on the release characteristics of the pharmaceutical formulation. It will also depend on the subject's health, size, weight, age, sex and tolerance to drugs. When administered more than once, the NPAR agonists can be administered at evenly spaced intervals. Each dose can be the same or different. A dose can be, for example, 0.1-500 μg, preferably 1-50 μg of NPAR agonist, and is commonly 3, 5, 10, 30 or 50 μg.

NPAR agonists can be administered by any suitable route, including by local introduction, for example, by endoscopy. The NPAR agonist can be administered intravenously. The NPAR agonist can be administered to the subject in a sustained release formulation, or can be delivered by a pump or an implantable device, or by an implantable carrier comprising polymers. “Administered to the smooth muscle” means delivered to the surface of the smooth muscle or into the smooth muscle itself. Alternatively, the point of delivery of the NPAR agonist can be in sufficient proximity to the surface of the smooth muscle so that the NPAR agonist can diffuse and contact the surface, for example, within 1 cm of the surface of a smooth muscle.

Compositions comprising the peptides can be administered by any suitable route, including orally, parentally, by inhalation spray, rectally, or topically in dosage unit formulations containing conventional pharmaceutically acceptable carriers, adjuvants, and vehicles. Parental administration includes subcutaneous, intravenous, intra-arterial, intramusclar, intrasternal, intratendinous, intraspinal, intracranial, intrathoracic, intraperitoneally, and by infusion techniques.

For parenteral application, particularly suitable vehicles consist of solutions, preferably oily or aqueous solutions, as well as suspensions, emulsions, or implants, including suppositories. Ampules are convenient unit dosages. It will be appreciated that the actually preferred amounts of active compounds used will vary according to the specific compound being utilized, the particular compositions formulated, the mode of application and the particular site of administration. Optimal administration rates for a given protocol of administration can be readily ascertained by those skilled in the art, using conventional dosage determination tests.

The NPAR agonists can be administered to the subject in conjunction with an acceptable pharmaceutical carrier as part of a pharmaceutical composition. The formulation of the pharmaceutical composition will vary according to the mode of administration selected. Suitable pharmaceutical carriers may contain inert ingredients which do not interact with the active compounds. The carriers should be biocompatible, i.e., non-toxic, non-inflammatory, non-immunogenic and devoid of other undesired reactions at the administration site. Examples of pharmaceutically acceptable carriers and other inert ingredients include, for example, saline, various buffers, alcohol, vegetable oils, polyethylene glycols, gelatin, lactose, sucrose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, etc. commercially available inert gels, or liquids supplemented with albumin, methyl cellulose or a collagen matrix. Further examples include sterile water, physiological saline, bacteriostatic saline (saline containing about 0.9% mg/ml benzyl alcohol), phosphate-buffered saline, Hank's solution, Ringer's-lactate and the like. Standard pharmaceutical formulation techniques can be employed, such as those described in Remington's Pharmaceutical Sciences (Remington's Pharmaceutical Sciences. 21^{st} edition, Mack Publishing Company, Easton, Pa. (2005)). The pharmaceutical preparations can be sterilized and if desired, mixed with auxiliary agents, e.g., lubricants, preservatives, stabilizers, wetting agents, emulsifiers, salts for influencing osmotic pressure, buffers, colorings and the like which do not deleteriously react with the active compounds.

Pharmaceutical compositions may include gels. Gels are compositions comprising a base selected from an oleaginous base, water, or an emulsion-suspension base. To the base is added a gelling agent which forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent or can be mixed after the gelation process.

In one embodiment, the NPAR agonists are administered in a sustained release formulation. Polymers are often used to form sustained release formulations. Examples of these polymers include poly α-hydroxy esters such as polylactic acid/polyglycolic acid homopolymers and copolymers, polyphosphazenes (PPHOS), polyanhydrides and poly (propylene fumarates).

Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson, et al., Adv. Drug Deliv. Rev. 28:5 (1997), the entire teachings of which are incorporated herein by reference). The incorporation of polyethylene glycol into the polymer as a blend to form microparticle carriers allows further alteration of the release profile of the active ingredient (see Cleek et al., J. Control Release 48:259 (1997), the entire teachings of which are incorporated herein by reference). Ceramics such as calcium phosphate and hydroxyapatite can also be incorporated into the formulation to improve mechanical qualities.

PPHOS polymers contain alternating nitrogen and phosphorous with no carbon in the polymer backbone, as shown below in Structural Formula (II):

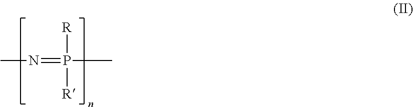

The properties of the polymer can be adjusted by suitable variation of side groups R and R′ that are bonded to the polymer backbone. For example, the degradation of and drug release by PPHOS can be controlled by varying the amount of hydrolytically unstable side groups. With greater incorporation of either imidazolyl or ethylglycol substituted PPHOS, for example, an increase in degradation rate is observed (see Laurencin et al., J Biomed Mater. Res. 27:963 (1993), the entire teachings of which are incorporated herein by reference), thereby increasing the rate of drug release.

In certain instances it may be advantageous to co-administer one or more additional pharmacologically active agents along with an NPAR agonist. Depending on the condition, co-administration with another therapeutic agent may be appropriate, for example, an anesthetic, an analgesic, a steroid, an anti-inflammatory agent, a benzodiazepene derivative, a thrombolytic agent such as tissue plasminogen activator (tPA), a blood thinning agent such as heparin.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these six articles are incorporated herein by reference in their entirety.

Thrombin peptide derivative dimers can be prepared by oxidation of the monomer (WO2004/005317). Thrombin peptide derivative dimers can be prepared by reacting the thrombin peptide derivative with an excess of oxidizing agent. A well-known suitable oxidizing agent is iodine.

A “subject” is preferably a human, but can also be an animal in need of treatment with a thrombin receptor agonist, e.g., companion animals (e.g., dogs, cats, and the like), farm animals (e.g., cows, pigs, horses and the like) and laboratory animals (e.g., rats, mice, guinea pigs and the like).

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

The term “aryl group” includes both carbocyclic and heterocyclic aromatic ring systems. Examples of aryl groups include phenyl, indolyl, furanyl and imidazolyl.

An “aliphatic group” is a straight chain, branched or cyclic non-aromatic hydrocarbon. An aliphatic group can be completely saturated or contain one or more units of unsaturation (e.g., double and/or triple bonds), but is preferably saturated, i.e., an alkyl group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. Aliphatic groups include, for example, methyl, ethyl, n-propyl, iso-propyl, n-butyl, sec-butyl, tert-butyl, pentyl, cyclopentyl, hexyl, cyclohexyl, octyl and cyclooctyl.

Suitable substituents for an aliphatic group, an aryl group or a non-aromatic heterocyclic group are those which do not significantly lower therapeutic activity of the NPAR agonist, for example, those found on naturally occurring amino acids. Examples include —OH, a halogen (—Br, —Cl, —I and —F), —O(Rₑ), —O—CO—(Rₑ), —CN, —NO₂, —COOH, ═O, —NH₂—NH(Rₑ), —N(Rₑ)₂. —COO(Rₑ), —CONH₂, —CONH(Rₑ), —CON(Rₑ)₂, —SH, —S(Rₑ), an aliphatic group, an aryl group and a non-aromatic heterocyclic group. Each Rₑ is independently an alkyl group or an aryl group. A substituted aliphatic group can have more than one substituent.

### EXAMPLE

### Effect of TP508 Pre-Treatment on Carbachol Induced Relaxation

Rat aortic rings (endothelial cell layer intact) were prepared. Rings were treated with no TP508 (control) or 1 mM TP508 for 1 hour. Rings were contracted with 500 nM norepinephrine, followed by increasing doses of carbachol (0, 1, 10, 100, 500, 750, 1000 and 5000 nM). Mean values±SEM are shown; n=2 animals.

Norepinephrine contracted rings, with intact endothelium, relax in response to increasing doses of carbachol (Furchgott, R. F. and Zawadzki, J. V., Nature 288: 373-376 (1980)). If TP508 is a smooth muscle relaxant, TP508 treatment prior to a norepinephrine-carbachol dosing regimen should result in an increase in carbachol induced relaxation compared to the control, i.e., a shift of the carbachol dose response curve to the left relative to the control curve.

The FIGURE demonstrates that at increased carbachol concentrations (greater than 250 nM; rectangular box) TP508 pre-treated rings showed increased relaxation relative to controls. Furthermore, TP508 pre-treatment leads to a sigmoidal carbachol dose response curve. The control rings produced a linear carbachol dose response curve.

While this invention has been particularly shown and described with references to example embodiments thereof, it will be understood by those skilled in the art that various changes in form and details may be made therein without departing from the scope of the invention encompassed by the appended claims.

1. A method for inducing relaxation of smooth muscle in a subject in need thereof, the method comprising administering to the subject a therapeutically effective amount of an agonist of a non-proteolytically activated thrombin receptor.

2-8. (canceled)

9. The method of claim 1, wherein the agonist is a thrombin peptide derivative comprising the amino acid sequence Asp-Ala-R, wherein R is a serine esterase conserved sequence, and the thrombin peptide derivative comprises from about 12 to about 23 amino acid residues.

10-16. (canceled)

17. The method of claim 1, wherein the thrombin peptide derivative comprises an N-terminus which is unsubstituted, and a C-terminus which is unsubstituted or a C-terminal amide represented by —C(O)NH₂.

18. (canceled)

19. The method of claim 17, wherein the thrombin peptide derivative comprises the polypeptide Arg-Gly-Asp-Ala-Cys-X₁-Gly-Asp-Ser-Gly-Gly-Pro-X₂-Val (SEQ ID NO:1), wherein X₁ is Glu or Gln and X₂ is Phe, Met, Leu, His or Val.

20-22. (canceled)

23. The method of claim 17, wherein the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative having at least eighteen amino acid residues, wherein X₁ is Glu or Gln and X₂ is Phe, Met, Leu, His or Val.

24. The method of claim 1, wherein the agonist is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

25-32. (canceled)

33. The method of claim 17, wherein the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5) or a fragment thereof comprising amino acid residues 10-18 of SEQ ID NO:5, wherein Xaa is alanine, glycine, serine or an S-protected cysteine; X₁ is Glu or Gln; and X₂ is Phe, Met, Leu, His or Val.

34-38. (canceled)

39. The method of claim 1, wherein the agonist is a peptide dimer comprising two thrombin peptide derivatives 12 to 23 amino acid residues in length which, independently, comprise the polypeptide Asp-Ala-Cys-X₁-Gly-Asp-Ser-Gly-Gly-Pro-X₂-Val (SEQ ID NO:10), wherein X₁ is Glu or Gln and X₂ is Phe, Met, Leu, His or Val, or a C-terminal truncated fragment thereof having at least six amino acid residues, provided that zero, one, two, or three amino acid residues in the polypeptide differ from those residues in the corresponding position of SEQ ID NO:10;
said thrombin peptide derivatives optionally comprising a C-terminal amide; and said thrombin peptide derivatives optionally comprising an acylated N-terminus;
the dimer is essentially free of monomer;
the thrombin peptide derivatives are the same;
the thrombin peptide derivatives are covalently linked through a disulfide bond; and
the thrombin peptide derivatives consist of from about 12 to about 23 amino acids.

40-44. (canceled)

45. The method of claim 39, wherein the thrombin peptide derivatives each comprise an N-terminus which is unsubstituted; and a C-terminus which is unsubstituted or a C-terminal amide represented by —C(O)NH₂.

46-49. (canceled)

50. The method of claim 45, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val or a fragment thereof comprising amino acid residues 10-18 of SEQ ID NO:2.

51. The method of claim 45, wherein the thrombin peptide derivatives comprise the polypeptide Ala-Gly-Tyr-Lys-Pro-Asp-Glu-Gly-Lys-Arg-Gly-Asp-Ala-Cys-Xᵢ-Gly-Asp-Ser-Gly-Gly-Pro-X₂-Val (SEQ ID NO:2), wherein Xᵢ is Glu or Gln and X₂ is Phe, Met, Leu, His or Val.

52-55. (canceled)

56. The method of claim 1, wherein the agonist is a peptide dimmer (SEQ ID NO: 3) represented by the following structural formula:

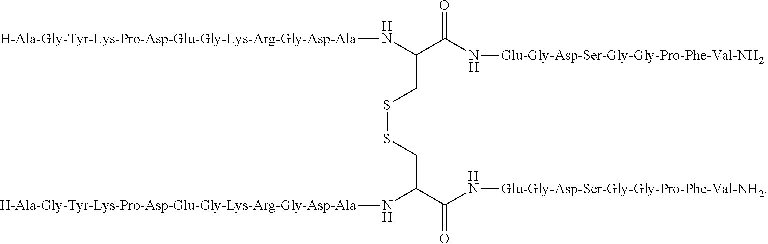

57. A method for causing relaxation of one or more sphincters in a subject, the method comprising administering to the subject a therapeutically effective amount of an NPAR agonist to the subject.

58. The method of claim 57, wherein the NPAR agonist causes relaxation of one or more of the following in the subject: the cardioesophageal sphincter, the gastroesophageal sphincter, the palatopharyngeal sphincter, the pharyngoesophageal sphincter, the pyloric sphincter, the internal rectal sphincter, the sphincter vaginae, and the tubal sphincter.

59. The method of claim 57, wherein the NPAR agonist causes relaxation of one or more sphincters in the subject to facilitate introduction of a medical device or medical instrument.

60. The method of claim 57, wherein the agonist is the polypeptide H-Ala-Gly-Tyr-Lys-Pro-Asp-Glu-Gly-Lys-Arg-Gly-Asp-Ala-Ser-Glu-Gly-Asp-Ser-Gly-Gly-Pro-Phe-Val-NH₂ (SEQ ID NO:28).
